(19) United States
(12) Patent Application Publication
(10) Pub. No.: US 2026/0232284 A1
(43) Pub. Date: Aug. 13, 2026

(54) COMPUTER-IMPLEMENTED METHOD FOR PROVIDING A TRAINED FUNCTION SET FOR DETERMINING A SCATTERED RADIATION INFORMATION ITEM, COMPUTER-IMPLEMENTED METHOD AND EVALUATING FACILITY FOR DETERMINING A SCATTERED RADIATION INFORMATION ITEM, COMPUTER PROGRAM, AND ELECTRONICALLY READABLE DATA CARRIER

(71) Applicant: Siemens Healthineers AG, Forchheim (DE)

(72) Inventors: Philipp Roser, Allston, MA (US); Noah Maul, Erlangen (DE)

(21) Appl. No.: 19/539,624

(22) Filed: Feb. 13, 2026

(30) Foreign Application Priority Data
Feb. 13, 2025 (DE) 10 2025 105 310.3

(51) Int. Cl. A61B 6/40; A61B 6/03; A61B 6/46

(52) U.S. Cl. CPC A61B 6/4014; A61B 6/032; A61B 6/466

(57) ABSTRACT

A method for providing a trained function set for determining a scattered radiation information item includes providing a first evaluating function that determines, from input data including an X-ray image, an average distribution dataset that defines an average scattered radiation distribution over a variation of an image recording parameter, as output data. A second evaluating function that determines, from input data including the image recording parameter during recording of the X-ray image, transformation parameters of a transformation as output data is provided, so that the scattered radiation information item is determinable by applying the transformation to the average distribution dataset. A training dataset is provided for each of a first number of basic scenarios for which only the image recording parameter is changeable. The method includes separate training of the evaluating functions, making use of the training datasets, and providing the trained first and second evaluating function.

This application claims the benefit of German Patent Application No. DE 10 2025 105 310.3, filed on Feb. 13, 2025, which is hereby incorporated by reference in its entirety.

### BACKGROUND

The present embodiments relate to a computer-implemented method for providing a trained function set for determining a scattered radiation information item defining a scattered radiation contribution in a two-dimensional X-ray image recorded with an X-ray facility. In addition, the present embodiments relate to a determining method, an evaluating facility, a computer program, and an electronically readable data carrier.

In X-ray imaging, scattered radiation may occur, for example, due to scattering processes on objects that are to be imaged and/or other transirradiated objects, which may also impinge upon the X-ray detector of the corresponding X-ray facility, and is measured accordingly. Such scattered radiation contributions are generally undesirable since such scattered radiation may hinder the evaluation of the corresponding X-ray image. A particularly negative influence comes from scattered radiation in cone beam computed tomography (CBCT) in which with a recording arrangement (e.g., a C-arm X-ray facility), projection images are recorded in various projection directions from which a three-dimensional X-ray image is then reconstructed. Without any suitable compensation, the scattered radiation may result in drastic impairments to the image quality of the reconstructed image dataset (e.g., to streak artifacts, cupping artifacts, and smearing artifacts). This may result in a more difficult or even a faulty evaluation.

For this reason, it is known from the prior art, in X-ray facilities, to arrange an anti-scatter grid in front of the X-ray detector, which is intended to restrict the incident X-ray radiation as far as possible to X-rays coming directly from the X-ray radiator by physically blocking X-ray beams from other directions. However, such anti-scatter grids also have disadvantageous effects.

First, the anti-scatter grid also unavoidably blocks a portion of the primary radiation so that an increase in the X-ray dose may potentially be required. Further, in two-dimensional X-ray imaging (e.g., therefore, in radiography), a better image quality and a lower dose may be achieved by removing the anti-scatter grid and using the air gap technique in which the X-ray detector is brought as close as possible to the object to be imaged. This applies, for example, for neurovascular imaging of the brain, for example, when investigating aneurysms and embolic strokes. In order to be able to provide X-ray facilities without anti-scatter grids that are also to be used for high quality two-dimensional imaging (e.g., of the brain), a software solution for scattered radiation correction is to be provided, at least for three-dimensional imaging processes (e.g., cone beam computed tomography).

Approaches have been proposed in the prior art in which machine learning is used in order to infer scattered radiation distributions from X-ray images. Although these procedures are extremely fast, their general robustness regarding the dependency upon the training data may be questionable. By reason of this dependency, it is difficult to unify all possible combinations of X-ray physics, collimation, focal spot properties, etc. in a combined training cohort. In addition, such functions may only be trained using simulated pairs of input and output data, so that a distinct gap remains between the simulation and reality.

It is known to determine scattered radiation distributions by simulating the imaging process, although for this, a great computation effort and relatively long calculation times are required.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a rapid and robust possibility for determining high quality scattered radiation information items in an X-ray facility is provided.

In a computer-implemented method for providing a trained function set for determining a scattered radiation information item defining a scattered radiation contribution in a two-dimensional X-ray image recorded with an X-ray facility, according to the present embodiments, the following acts are provided: providing a first evaluating function that is to be trained that determines, from input data including the X-ray image, an average distribution dataset that defines an average scattered radiation distribution over a variation of at least one image recording parameter, as output data; providing a second evaluating function that is to be trained that determines, from input data including the at least one image recording parameter during recording of the X-ray image, transformation parameters of a transformation as output data so that the scattered radiation information item may be determined by applying the transformation to the average distribution dataset; providing a training dataset for each of a first number of basic scenarios for which only the at least one image recording parameter is changeable, where each training dataset for different values of the at least one image recording parameter includes a second number of subdatasets, each including a training X-ray image as input data for the first evaluating function, a training value set of the at least one image recording parameter as input data for the second evaluating function and a training scattered radiation image defining the scattered radiation distribution as a respective ground truth; separate training of the evaluating functions making use of the training datasets; and providing the trained first and second evaluating function.

The present embodiments may be used in medical imaging technology. In the energy range of the X-ray radiation used for diagnostic or general imaging, X-ray scattered radiation is dominated by components of low spatial frequency. Although high frequency noise also exists, for three-dimensional imaging at least, the low-frequency component is the most problematic, for example, with regard to cone beam computed tomography (CBCT). But also, in general, the scattered radiation distribution and/or the scattered radiation contribution (e.g., scattered radiation signal) for a two-dimensional X-ray image recorded as a projection may be understood to be a low-frequency surface.

The present embodiments are based upon the finding that the general form of the scattered radiation distribution for different imaging parameters is similar, provided the same recording geometry and the same imaging object are present, for example, as the recorded region of a human body. This process has been established for a large number of image recording parameters. Therefore, it may be provided, for example, that the basic scenarios relate to the recording of a specified imaging object in a specified recording geometry and/or the at least one image recording parameter is selected from the group including a tube current of an X-ray radiator of the X-ray facility, a tube voltage of the X-ray radiator, a focal spot size, and a type of focus of the focal spot of the X-ray radiator, at least one filter parameter defining a filter measure for the X-ray radiation, and at least one modulation parameter relating to a modulation of the X-ray radiation. Therein, the X-ray radiator, for example, is configured as or includes an X-ray tube.

Based upon the aforementioned finding, it is provided to subdivide the X-ray scattered radiation model into two portions, specifically into an average distribution dependent upon the imaging object (e.g., the anatomy, such as an average shape), and into a variation dependent upon the image recording parameters, defined by a transformation of the average distribution. Herein, the transformation may include the weighted application of basic variation components that may also be designated variation basis components, where the transformation parameters define the weighting. This concept is comparable to statistical shape models that may often be used to model objects (e.g., organs). In this regard also, an average shape is assumed that is varied in order to define a specific instance.

For the average distribution dataset, a representation (e.g., a model) of the average scattered radiation distribution may be used (e.g., a vertex-based and/or spline-based surface as an average shape and/or an implicit function and/or a parametric function). Herein, a scattered radiation distribution may be differently modeled and may thus exist in a different representation. Dependent upon the representation of the average distribution (e.g., the average shape of the scattered radiation distribution), the transformation and, for example, the basic variation components may assume different forms. For example, it may be provided that for a surface as the representation, the basic variation components include displacement vectors. A surface may be a vertex-based mesh and/or a spline-based surface (e.g., described by spline functions, such as B-splines). For a convolution as the transformation that may be applied, for example, to functions, the basic variation components may define a standard convolution. If the transformation is a matrix multiplication, the basic variation components may be matrix-based basis components.

Overall, the basic variation components may serve or be understood as a basis for the transformation and therefore may also be designated variation basis components. For example, for an average shape of the scattered radiation distribution represented by a surface, the transformation may include a linear combination of the average shape defined by the average distribution dataset and a basis of eigenvectors weighted with the transformation parameters as the basic variation components.

This subdivided representation of the scattered radiation distribution is combined, according to the present embodiments, with a scattered radiation estimate on the basis of machine learning. In place of the scattered radiation distribution, that is, to determine the scattered radiation contribution solely on the basis of the X-ray image, this approach also enables image recording parameters to be taken into account. This adds physical plausibility to the scattered radiation estimate based upon machine learning, enables useful incorporation of image recording parameters in the inference, and improves training data efficiency. In addition, as is considered in greater detail below, safety mechanisms for the inference are determined and used in order to warn in the event of implausible scattered radiation information and/or to take other measures.

With regard to machine learning, in the present case, two trained evaluating functions are trained and provided. The first trained evaluating function determines as output data the average distribution dataset, including for any desired actual scenarios, for example, defined by the imaging object and a recording geometry. For this purpose, the first trained evaluating function is trained such that, at least for the basic scenarios for which training datasets are available, it correctly reproduces the average distribution defined by them. For this purpose, the first trained evaluating function may exclusively use the X-ray image.

The second trained evaluating function uses the image recording parameters in order to deduce transformation parameters therefrom that ultimately define what influence the image recording parameters exert and how, on the basis of the selection of the image recording parameters, that is, their values that are to be modified for the scenario by the average distribution defined by the average distribution dataset in order to determine the most accurate possible scattered radiation distribution for the X-ray image. Since, based on the subdivision undertaken into the portions prescribed by the scenario and the portions prescribed by the image recording parameters, both trained evaluating functions use different input data and different output data, they may be trained separately and each ignore a part of the maximum parameter space to be covered (e.g., various degrees of freedom that cover the respective other evaluating function, so that fewer complex, more robust learning processes, possibly also needing less training data, are enabled). Therein, specific architectures and sequences for training the first and second evaluating functions are considered below in greater detail.

The function set provided may relate to an application area wherein the quantity of training data needed may be selected according to the field of use (e.g., the variability contained therein). If the application area is, for example, neurovascular imaging of the human head, there is relatively little variation there (e.g., also with the image recording parameters), so that, for example, for each basic scenario, 5 to 100 subdatasets (e.g., 50 subdatasets) may be sufficient. The first count of the basic scenarios may also vary with respect to the application area; in neurovascular imaging, for example, a first count of 10 to 100 (e.g., again 50) has been found to be suitable. For other application areas in which there is more variability, greater first and second counts may be useful.

In general, a trained function maps cognitive functions that humans associate with other human brains. Via training based upon training data (e.g., machine learning), the trained function is capable of adapting itself to new circumstances and of detecting and extrapolating patterns. Another expression for “trained function” is “trained machine learning model.”

In general, parameters of a trained function may be adapted via training. For example, supervised learning, semi-supervised learning, unsupervised learning, reinforcement learning, and/or active learning may be used. In addition, representation learning (e.g., also known as “feature learning”) may be used. The parameters of the trained function may be adapted, for example, iteratively via a plurality of training steps. For example, with the training, a particular cost function may be minimized. For example, during training of a neural network, the back propagation algorithm may be utilized.

A trained function may include, for example, a neural network, a support vector machine (SVM), a decision tree, and/or a Bayesian network, and/or the trained function may be based upon k-means clustering, Q-learning, genetic algorithms, and/or assignment rules. For example, a neural network may be a deep neural network, a convolutional neural network (CNN), or a deep CNN. Further, the neural network may be an adversarial network, a deep adversarial network, and/or a generative adversarial network (GAN).

A convolutional neural network (CNN) is a neural network that uses a convolution operation rather than a general matrix multiplication in at least one of its layers, the convolutional layer. For example, a convolutional layer may perform a scalar product of one or more convolution kernels with the associated data/images of the convolutional layer, where the entries of the one or the plurality of convolution kernels are the parameters or weights that are adapted through training. For example, the inner Frobenius product and the ReLu activation function may be used. A CNN may include additional layers, for example, pooling layers, fully connected layers, and normalization layers.

By way of CNNs, input images may be processed extremely efficiently since a convolution operation based on different kernels may extract the most widely differing image features so that, by adapting the weights of the convolution kernel, the relevant image features may be found during the training. Further, based on the sharing of the weights in the convolutional layer kernels, fewer parameters have to be trained so that an overfitting in the training phase is prevented and rapid training or a larger number of layers are allowed in the CNN, so that the efficiency of the network is increased. To this extent, in the context of this disclosure, the use of at least one CNN suggests itself, including at least for the first trained evaluating function (e.g., also for the second trained evaluating function).

Specifically, in the context of this disclosure, it may be provided that the training scattered radiation images are determined, at least partially (e.g., completely) via simulation. While it is also conceivable, in principle, in admittedly complex measuring processes, for example, using phantoms, to determine scattered radiation images by way of measurements, according to the present embodiments, simulated X-ray scattered radiation images may be used as training scattered radiation images. The training X-ray images may also be the result of a simulation. However, it may also be provided that the training X-ray images are determined at least partially via a recording process where for simulation of an associated training scattered radiation image, image recording parameters of the recording process are used. Corresponding simulation methods are already widely known from the prior art and may accordingly also be used for determining training data in the context of this disclosure. Training scattered radiation images may naturally be converted without difficulty into each suitable specific representation that is to be used for the average distribution dataset.

In a development of the present embodiments, it may be provided that the image recording parameters are provided as an X-ray spectrum of the X-ray radiation and/or a spatial distribution for modeling the focal spot. Thus, primary image recording parameters such as, for example, tube voltage, tube current, and suchlike or focus type and focal spot size are mapped in a continuous distribution also covering other properties of the X-ray facility, so that the quantity of the information provided is suitably increased. In other words, more physical knowledge is introduced, and this increases the quality of the scattered radiation information that may be established with the function set.

Suitably, training output datasets may be determined as a ground truth for training at least one of the evaluating functions from the training datasets that are used for supervised learning of the respective evaluating function. In other words, the training data provided by the training datasets (e.g., the training scattered radiation images) also define the output data that is provided for the first evaluating function and the second evaluating function, so that it may suitably be derived as a ground truth from the training data for at least one of the evaluating functions.

For example, it may be specifically provided that for each basic scenario, a training average distribution dataset is determined from the scattered radiation distributions that are defined by the training scattered radiation images of the subdatasets, for example, via average formation and/or generalized Procrustes analysis, where the first evaluating function is trained with corresponding training X-ray images of all the subdatasets of all the training datasets. A training average distribution dataset may thus be determined, for example, by average formation and/or Procrustes analysis from the scattered radiation distributions for the respective basic scenarios so that then all training X-ray images of the training datasets may be used for training on the respective training average distribution dataset as the ground truth. However, it is also fundamental, as will be considered in greater detail, that an indirect determination of output average distribution datasets for the respective basic scenarios via semi-supervised learning is possible.

Suitably, when the output average distribution datasets are determined for each basic scenario, the transformation and the training transformation parameters to be used as a ground truth may be determined from the output average distribution datasets and the training scattered radiation images. The output average distribution datasets may be the training average distribution datasets, but may also be otherwise determined. In other words, with regard to the transformation and the ground truths relating to the transformation parameters, a variation analysis may be carried out. In this regard, it may be suitable to provide a harmonization step that has the result that for all the basic scenarios and thus also the later general scenario space for which the function set is to be applied, the same transformation is available. Therefore, an identical transformation is provided for all scenarios.

For example, if the transformation includes the weighted application of basic variation components, where the transformation parameters define the weighting, the determination may include determining the weighting of the basic variation components. The basic variation components then define the transformation. In general, it may be stated that, in the use of basic variation components, known procedures may be utilized in order to determine these as the basis for the transformation. For example, it may be provided that the basic variation components of the transformation and the training transformation parameters are determined making use of a principal component analysis (PCA) and/or an independent component analysis (ICA) and/or using a variational autoencoder (VAE). Any other possibilities for deriving a representation of variations starting from an average distribution may naturally also be utilized in the context of the present invention.

If training transformation parameters are determined, the second evaluating function may be trained directly with the respective pairs of image recording parameter value sets and transformation parameters. A value set for one single image recording parameter may also contain just one value; in the context of the present embodiments, however, a plurality of imaging parameters (e.g., their values) are used as input data for the second evaluating function.

In a development of the present embodiments, it may be provided that during the training, at least one statistical value defining the distribution of the transformation parameters is determined, for example, per transformation parameter, from which statistical value at least one safety condition for plausibility checking of the transformation parameters that are output by the second evaluating function is determined and is provided therewith. Therefore, safeguards or “guardrails” may be provided in the inference by assessing the statistical distribution of the transformation parameters during the training or its conclusion in order to be able to stipulate plausible results of the second evaluating function.

The safety condition thus indicates, for example, on its fulfilment, that the corresponding at least one transformation parameter that it evaluates lies in an expected range whereas, on non-fulfilment of the safety condition, the presence of an unusual, unexpected result is indicated. A design of this type is particularly suitable if the subdatasets for at least one of the at least one recording parameter cover an entire settable range. If, for example, a tube voltage of between 70 and 175 kV may be selected, the boundary values may also be covered, and it is known that the most scattered radiation is also expected at the highest tube voltage. An expected range may therefore be derived from extremes of transformation parameters determined at least substantially by the tube voltage. In general terms, the at least one statistical value may include, for example, a minimum and/or a maximum and/or a mean value and/or a median and/or a standard deviation.

In the provision of the at least one safety condition for the second evaluating function, therefore, in the course of the inference, it may be provided that on non-fulfilment of at least one of the at least one safety condition, a notification output to the user takes place (e.g., a confirmation is requested), although to continue with the output data of the second evaluating function, and/or a, for example, more robust, fallback method for determining the scattered radiation information is used. Thus, during the training, extremes and/or other statistically relevant statistical values relating to the transformation parameters are recorded. These statistical values are used in the context of the inference as a safety mechanism. If, during the inference, improbable transformation parameters (e.g., therefore, improbable weights) are determined, the end user may be warned rather to check the results again and/or a return to a less accurate, but possibly more robust fallback methodology for determining the scattered radiation information may take place. For example, despite the longer duration as the fallback methodology, a simulation of the scattered radiation distribution or suchlike may take place.

In principle, a large number of possibilities for specific implementation of the training exist. In a first variant, as described, ground truths for the respective output data of the evaluating functions are determined from the training datasets, after which a supervised learning of the first and the second evaluating function may take place. In one variant, it may also be provided not to determine the training transformation parameters explicitly, but rather, in effect, to attach the relevant average distribution dataset and the transformation in order to use the training scattered radiation images directly as a ground truth.

However, variations may also be provided in which the first and/or the second evaluating function are trained semi-supervised. Thus, for example, it may be provided that the first evaluating function is trained (e.g., in terms of basic scenarios) with all the conceivable pairings of input X-ray images and training scattered radiation images as a ground truth. Therein, use is made of the fact that trained functions of the machine learning implicitly act statistically. If the first evaluating function attempts to represent all the training scattered radiation images for a particular training X-ray image as well as possible, an average of these training scattered radiation images arises as an average distribution dataset in a natural manner, for example, using the mean square error. The averaging therefore implicitly takes place in the learning process.

A suitable development of the present embodiments may further provide that in a transformation including the weighted application of basic variation components in the training process for the second evaluating function, the basic variation components are also used as trainable parameters. In this way, the basic variation components and the second evaluating function may implicitly be trained taking account of the respective average distribution dataset and the training scattered radiation images. The basic variation components are thus interpreted as a trainable parameter set. In this way, a common transformation for all the basic scenarios may also implicitly be found. Specifically, for example, it may be provided that the transformation is modeled as a plurality of trained transformation functions (e.g., neural networks), where each basic variation component is associated, for example, with exactly one trained transformation function. For example, the transformation as a transformation module may be a “forest” of neural networks, of which the count corresponds to the number of variation modules that may be included in the training process. It should be noted in this regard that it is naturally also conceivable to use at least one parameter defining the basic variation components itself as a transformation parameter. For example, the transformation may provide a basic form of the basic variation components, whereas the specific selection thereof is made case-dependently via transformation parameters. In one embodiment, however, as described, to select a fixed transformation with fixed basic variation components, the weights of which are then defined by the transformation parameters, taking account of the representation of the scattered radiation distribution in the average distribution dataset.

Procedures are also conceivable in which the second evaluating function is pre-trained and, during training of the first evaluating function, based on the training scattered radiation image as a ground truth, is kept unchangeable (“frozen”). Then, for example, it may be provided that first the second evaluating function is trained based on established training average distribution datasets and making use of the transformation, where the resultant scattered radiation distribution is compared with the training scattered radiation images as a ground truth. Thereafter, the first evaluating function is also trained in the overall complex with the training scattered radiation images as a ground truth and the unchangeably trained second evaluating function.

Apart from the providing method, the present embodiments also relate to a computer-implemented method for determining a scattered radiation information item defining a scattered radiation contribution in a two-dimensional X-ray image recorded with an X-ray facility, including the following acts. A trained function set (e.g., using a providing method according to the present embodiments) is provided. The function set includes: a first trained evaluating function that determines, from input data including the X-ray image, an average distribution dataset that defines an average scattered radiation distribution over a variation of at least one image recording parameter, as output data; a second trained evaluating function that determines, from input data including the at least one image recording parameter during recording of the X-ray image, transformation parameters of a transformation as output data so that the scattered radiation information item may be determined by applying the transformation to the average distribution dataset; providing the X-ray image to be evaluated and the at least one image recording parameter associated with it; applying the first trained evaluating function to the X-ray image to be evaluated as input data for determining an average distribution dataset associated with the X-ray image to be evaluated, as output data; applying the second trained evaluating function to the at least one image recording parameter associated with the X-ray image to be evaluated as input data for determining transformation parameters associated with the X-ray image to be evaluated, as output data; determining the scattered radiation information item, for example, in the form of a scattered radiation image by applying the transformation according to the transformation parameters associated with the X-ray image to be evaluated to the average distribution dataset associated with the X-ray image to be evaluated.

All the embodiments relating to the providing method according to the present embodiments may be transferred similarly to the determining method according to the present embodiments and vice versa, so that the advantages mentioned above may therefore also be obtained with the determining method.

For example, it may thus be provided that on provision of at least one safety condition to the second evaluating function and on non-fulfilment of at least one of the at least one safety condition, a notification output to the user takes place (e.g., a confirmation is requested), although to continue with the output data of the second evaluating function, and/or a, for example, more robust, fallback method for determining the scattered radiation information is used.

In general, it is thus provided in the determining method according to the present embodiments for determining the scattered radiation information item that may be provided as a scattered radiation image, not only is the X-ray image used, but also image recording parameters relating to the X-ray image. In the recording process, therefore, not only is the X-ray image stored, but also the values of the image recording parameters used that are associated with it. The X-ray image is transferred to the first evaluating function as input data, while the value set of the image recording parameters associated with the X-ray image is passed to the second evaluating function. In this way, first, an average distribution of the scattered radiation is determined by the first evaluating function, and second, transformation parameters (e.g., weights) for the transformation by the second evaluating function. This enables the scattered radiation distribution for the X-ray image to be determined. Herein, as mentioned above, safety conditions are to be taken into account.

Thereby, a rapid, efficient, exact, robust and physically plausible determination of scattered radiation distributions for X-ray images is enabled. The scattered radiation information item may be used, for example, for correcting the X-ray image for scattered radiation contributions. The X-ray images may be, for example, projection images of a three-dimensional imaging process (e.g., from cone beam computed tomography). The determining method may naturally also be used for other two-dimensional transirradiation X-ray images (e.g., radiography and/or fluoroscopy images).

In a suitable development of the determining method, it may be provided that for a subsequent recording process with the same imaging object and the same recording geometry, in an optimizing process making use of the second evaluating function with variation of the at least one image recording parameter, an optimum set of the at least one image recording parameter is determined for a predetermined optimization goal and is utilized, for example, for driving the X-ray facility in the subsequent recording process. Using the function set, it is therefore possible to undertake an analysis of expected scattered radiation distributions for different configurations of the recording parameters in order to determine optimum image recording parameters with regard to the scattered radiation before the recording of further X-ray image data. Herein, for example, it may be provided that the optimization goal relates to a general or locally limited minimization of the scattered radiation. If a particular region of the X-ray image to be recorded is to be affected as little as possible by scattered radiation, the optimization goal may aim, for example, at a minimization in this region.

It should be noted that if, in some manner, the average distribution of the scattered radiation may also be determined for a particular current scenario even without prior recording of an X-ray image, it is also conceivable to carry out this optimization before the recording of the first X-ray image.

An evaluating facility according to the present embodiments may be a computing facility with at least one processor. The evaluating facility for determining a scattered radiation information item defining a scattered radiation contribution in a two-dimensional X-ray image recorded with an X-ray facility has a storage device in which a trained function set is stored, the function set including: a first trained evaluating function that determines, from input data including the X-ray image, an average distribution dataset that defines an average scattered radiation distribution over a variation of at least one image recording parameter, as output data; a second trained evaluating function that determines, from input data including the at least one image recording parameter during recording of the X-ray image, transformation parameters of a transformation as output data so that the scattered radiation information item may be determined by applying the transformation to the average distribution dataset; an interface for receiving the X-ray image to be evaluated and the at least one image recording parameter associated with it; a first applying unit for applying the first trained evaluating function to the X-ray image to be evaluated as input data for determining an average distribution dataset associated with the X-ray image to be evaluated, as output data; a second applying unit for applying the second trained evaluating function to the at least one image recording parameter associated with the X-ray image to be evaluated as input data for determining transformation parameters associated with the X-ray image to be evaluated, as output data; and a determining unit for determining the scattered radiation information item, for example, in the form of a scattered radiation image by applying the transformation according to the transformation parameter associated with the X-ray image to be evaluated to the average distribution dataset associated with the X-ray image to be evaluated.

For the evaluating facility also, the statements made above regarding the providing method and the determining method apply accordingly. The evaluating facility thus permits the achievement of the aforementioned advantages.

The evaluating facility has at least one processor and the storage device. Functional units that permit the carrying out of acts of the determining method according to the present embodiments are formed via hardware and/or software. Apart from the applying unit and the first or second determining unit, they may also include further functional units (e.g., a training unit for training the evaluating function or thus for carrying out a providing method according to the present embodiments). The evaluating facility may also have a correcting unit for applying the scattered radiation information item to the X-ray image for correcting the X-ray image.

The evaluating facility may be provided as part of an imaging X-ray facility (e.g., integrated into its control facility), for example, in order to be able to correct recorded X-ray images with regard to the scattered radiation (e.g., in the context of a three-dimensional imaging process directly on site). For example, the X-ray image may be a projection image of a cone beam computed tomography recording. If each projection image is now corrected with regard to scattered radiation, thereafter, the reconstruction of a three-dimensional image dataset of enhanced quality with, for example, a reduced artifact count may take place.

In one embodiment, an X-ray facility that includes an evaluating facility according to the present embodiments is provided. The X-ray facility also includes an X-ray radiator, an X-ray detector, and a control facility (e.g., a controller). An X-ray image recorded with the X-ray facility making use of particular image recording parameters may be evaluated by the evaluating facility together with the utilized values of the image recording parameters for determining the associated scattered radiation information and, for example, corrected. The X-ray facility may be, for example, an X-ray facility with a C-arm on which the X-ray radiator and the X-ray detector are arranged opposite one another. Such X-ray facilities are often used in angiography and/or for monitoring, for example, minimally invasive medical interventions. For example, the X-ray facility may be used for neurovascular imaging. X-ray images recorded diagnostically and/or accompanying the intervention (e.g., projection images of a cone beam computed tomography may be corrected for scattered radiation contributions directly on site before display on a display facility and/or their use for reconstruction of a higher-dimensioned image dataset). The X-ray facility has, for example, no anti-scatter grid.

Similarly to the evaluating facility that is configured for carrying out a determining method according to the present embodiments, a providing method may also be provided that is configured or carrying out a providing method according to the present embodiments. The providing facility is also a computing facility with at least one processor and at least one storage device. The providing facility for providing a trained function set for determining a scattered radiation information item defining a scattered radiation contribution in a two-dimensional X-ray image recorded with an X-ray facility may include: a first interface for receiving a first evaluating function that is to be trained that determines, from input data including the X-ray image, an average distribution dataset that defines an average scattered radiation distribution over a variation of at least one image recording parameter, as output data; a second interface for receiving a second evaluating function that is to be trained that determines, from input data including the at least one image recording parameter, during recording of the X-ray image, transformation parameters of a transformation as output data so that the scattered radiation information item may be determined by applying the transformation to the average distribution dataset; a third interface for receiving a training dataset for each of a first number of basic scenarios for which only the at least one image recording parameter is changeable, where each training dataset for different values of the at least one image recording parameter includes a second number of subdatasets, each including a training X-ray image as input data for the first evaluating function, a training value set of the at least one image recording parameter as input data for the second evaluating function, and a training scattered radiation image including the scattered radiation distribution as a respective ground truth; a training unit for separate training of the evaluating functions making use of the training datasets; and a fourth interface for providing the trained first and second evaluating function.

A computer program according to the present embodiments is able to be loaded directly into a storage device of a computing facility of an evaluating facility and/or a providing facility and has program means such that when the computer program is executed on the evaluating facility, the evaluating facility is caused to carry out a determining method and/or a providing method according to the present embodiments. The computer program may thus be a determining computer program, a providing computer program, and/or a combined determining and providing computer program. The computer program may be stored on an electronically readable data carrier (e.g., a non-transitory computer-readable storage medium) according to the present embodiments that therefore includes control information stored thereon, that comprises at least a computer program according to the present embodiments and is configured such that, on use of the data carrier in a computing facility, the computing facility is configured to carry out a method according to the present embodiments. The computing facility may be a providing facility and/or an evaluating facility dependent upon whether a determining method and/or a providing method is to be carried out. The data carrier may be, for example, a non-transient data carrier (e.g., a CD-ROM).

### BRIEF DESCRIPTION OF THE DRAWINGS

Further advantages and details of the present invention are disclosed in the example embodiments described below, making reference to the drawings. In the drawings:

FIG. 1 shows an example embodiment of a neural network;

FIG. 2 shows an example embodiment of a convolutional neural network;

FIG. 3 shows schematically, scattered radiation properties of two X-ray images of the same scenario;

FIG. 4 shows a representation illustrating the subdivision of scattered radiation distributions in an average distribution and of a variation;

FIG. 5 shows a diagram of the flow of a determining method according to an embodiment;

FIG. 6 shows a flow diagram of an example embodiment of a providing method according to an embodiment;

FIG. 7 shows a diagram for determining ground truths and a transformation from training data;

FIG. 8 shows the functional structure of an evaluating facility according to an embodiment; and

FIG. 9 shows a conceptual sketch of an X-ray facility.

### DETAILED DESCRIPTION

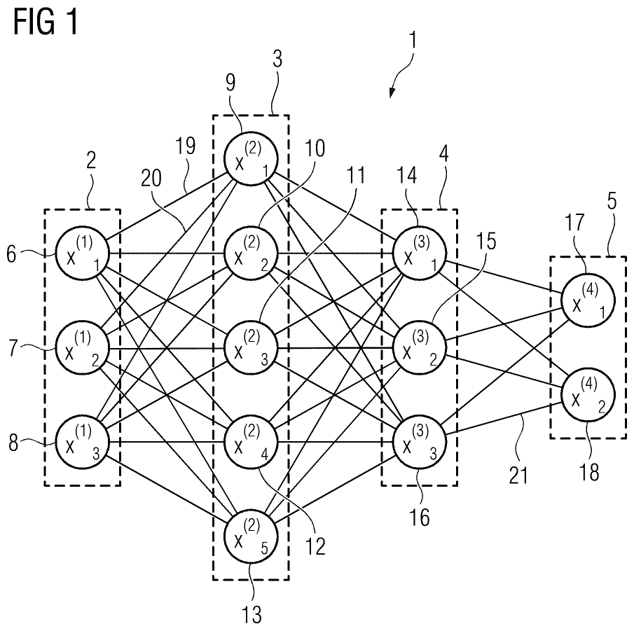

FIG. 1 shows an example embodiment of an artificial neural network 1. Other expressions for the artificial neural network 1 are “neural network”, “artificial neural net” or “neural net”.

The artificial neural network 1 comprises nodes 6 to 18 and edges 19 to 21, wherein each edge 19 to 21 is a directed connection from a first node 6 to 18 to a second node 6 to 18. In general, the first node 6 to 18 and the second node 6 to 18 are different nodes 6 to 18, although it is also conceivable that the first node 6 to 18 and the second node 6 to 18 are identical. For example, in FIG. 1, the edge 19 is a directed connection from the node 6 to the node 9 and the edge 21 is a directed connection from the node 16 to the node 18. An edge 19 to 21 from the first node 6 to 18 to a second node 6 to 18 is designated an “ingoing edge” for the second node 6 to 18 and as an “outgoing edge” for the first node 6 to 18.

In this example embodiment, the nodes 6 to 18 of the artificial neural network 1 may be arranged in layers 2 to 5, wherein the layers may have an intrinsic order which is introduced by the edges 19 to 21 between the nodes 6 to 18. In particular, edges 19 to 21 may only be provided between adjacent layers of nodes 6 to 18. In the example embodiment shown, there exists an input layer 2 which has only the nodes 6, 7, 8, in each case without an ingoing edge. The output layer 5 comprises only the nodes 17, 18 each without outgoing edges, wherein furthermore, hidden layers 3 and 4 lie between the input layer 2 and the output layer 5. In the general case, the number of hidden layers 3, 4 may be selected arbitrarily. The number of nodes 6, 7, 8 of the input layer 2 typically corresponds to the number of input values into the neural network 1 and the number of the nodes 17, 18 in the output layer 5 typically corresponds to the number of the output values of the neural network 1.

In particular, a (real) number may be allocated to the nodes 6 to 18 of the neural network 1. Therein, x⁽ⁿ⁾ᵢ denotes the value of the i-th node 6 to 18 of the n-th layer 2 to 5. The values of the nodes 6, 7, 8 of the input layer 2 are equivalent to the input values of the neural network 1, while the values of the nodes 17, 18 of the output layer 5 are equivalent to the output values of the neural network 1. Furthermore, each edge 19, 20, 21 may be allocated a weight in the form of a real number. In particular, the weight is a real number in the interval [−1, 1] or in the interval [0, 1]. Therein, w^{(m,n)}ᵢⱼ denotes the weight of the edge between the i-th nodes 6 to 18 of the m-th layer 2 to 5 and the j-th nodes 6 to 18 of the n-th layer 2 to 5. (n,n+1) Furthermore, the abbreviation

$${w}_{i , j}^{( n )}$$

is defined for the weight

$${w}_{i , j}^{( n , n + 1 )} .$$

In order to calculate output values of the neural network 1, the input values are propagated by the neural network 1. In particular, the values of the nodes 6 to 18 of the (n+1)-th layer 2 to 5 may be calculated on the basis of the values of the nodes 6 to 18 of the n-th layer 2 to 5 with

$${x}_{j}^{( n + 1 )} = f ( {\sum }_{i} {x}_{i}^{( n )} \cdot {w}_{i , j}^{( n )} )$$

Therein, f is a transfer function which may be designated the activation function. Known transfer functions are step functions, sigmoid functions (for example, the logistic function, the generalized logistic function, the hyperbolic tangent, the arctangent, the error function, the smoothstep function) or rectifiers. The transfer function is substantially used for normalizing purposes.

In particular, the values are propagated layerwise by way of the neural network 1, wherein values of the input layer 2 are given by way of the input data of the neural network 1. Values of the first hidden layer 3 may be calculated on the basis of the values of the input layer 2 of the neural network 1, and values of the second hidden layer 4 may be calculated on the basis of the values in the first hidden layer 3, etc.

In order to be able to specify the values

$${w}_{i , j}^{( n )}$$

for the edges 19 to 21, the neural network 1 must be trained using training data. In particular, training data comprises training input data and training output data which is denoted below as ti. For a training step, the neural network 1 is applied to the training input data in order to determine calculated output data. In particular, the training output data and the calculated output data comprises a number of values, wherein the number is determined as the number of the nodes 17, 18 of the output layer 5.

In particular, a comparison between the calculated output data and the training output data is used to adapt recursively the weights within the neural network 1 (“back-propagation algorithm”). In particular, the weights may be amended according to

$${w}_{i , j}^{′ ( n )} = {w}_{i , j}^{( n )} - γ \cdot {δ}_{j}^{( n )} \cdot {x}_{i}^{( n )}$$

wherein γ is a learning rate and the numbers

$${δ}_{j}^{( n )}$$

may be calculated recursively according to

$${δ}_{j}^{( n )} = ( {\sum }_{k} {δ}_{k}^{( n + 1 )} \cdot {w}_{j , k}^{( n + 1 )} ) \cdot f ( {\sum }_{i} {x}_{i}^{( n )} \cdot {w}_{i , j}^{( n )} )$$

on the basis of

$${δ}_{j}^{( n + 1 )}$$

if the (n+1)-th layer is not the output layer 5, and

$${δ}_{j}^{( n )} = ( {x}_{j}^{( n + 1 )} - {t}_{j}^{( n + 1 )} ) \cdot {f}^{′} ( {\sum }_{i} {x}_{i}^{( n )} \cdot {w}_{i , j}^{( n )} )$$

if the (n+1)-th layer is the output layer 5, wherein f′ is the first derivative of the activation function and

$${t}_{j}^{( n + 1 )}$$

is the comparative training value for the j-th nodes 17, 18 of the output layer 5.

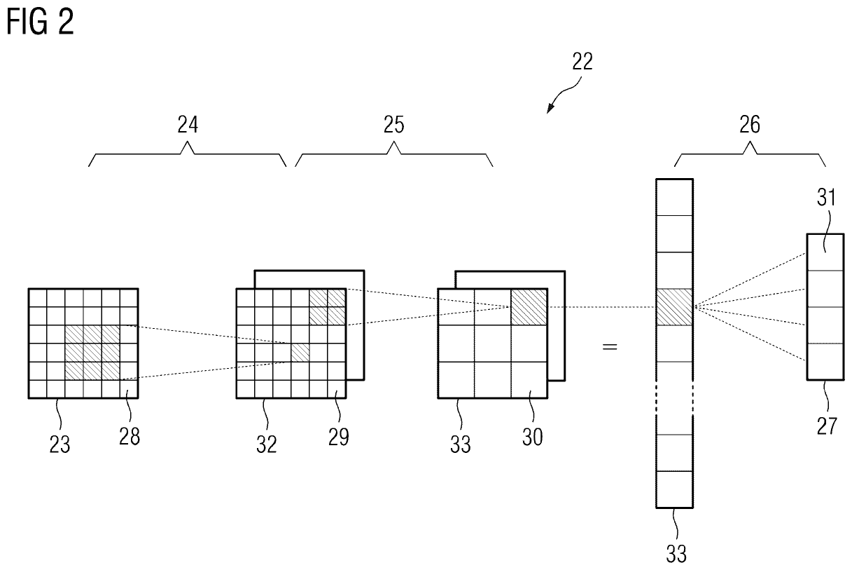

An example will now also be given for a convolutional neural network (CNN), making reference to FIG. 2. It should be noted here that the expression “layer” is often understood here in two different ways. Firstly, the expression “layer” relates to the set of nodes that is connected to other such groups of nodes via ingoing and/or outgoing edges. Secondly, the expression “layer” is understood as two groups of nodes that are connected by way of a specific structure of edges (e.g. convolutional layer, pooling layer, fully connected layer) so that each group of nodes is associated with two such “layers”. For differentiation, the first type of layers is designated node layers and the second type (which is not otherwise clear from the name) as connected layers.

FIG. 2 shows an example embodiment of a convolutional neural network 22. In the example embodiment shown, the convolutional neural network 22 comprises an input layer 23, a convolutional layer 24, a pooling layer 25, a fully connected layer 26 and an output node layer 27, as well as hidden node layers 32, 33. In alternative embodiments, the convolutional neural network 22 may contain a plurality of convolutional layers 24, a plurality of pooling layers 25 and a plurality of fully connected layers 26, just like other types of layers. The sequence of the layers may be selected as desired, wherein typically, fully connected layers 26 form the last layers before the output layer 27.

In particular, within a convolutional neural network 22, the nodes 28 to 30 of one of the node layers 23, 32 to 33 may be understood as being arranged in a d-dimensional matrix or as a d-dimensional image. In particular, in the two-dimensional case, the value of a node 28 to 30 may be denoted with the indices i, j in the n-th node layer 23, 32, 33 as x⁽ⁿ⁾ [i,j]. It should be noted that the arrangement of the nodes 28 to 30 of a node layer 23, 32, 33 has no effect on the calculations within the convolutional neural network 22 as such, since these effects are produced exclusively by the structure and the weights of the edges.

A convolutional layer 24 is distinguished, in particular, in that the structure and the weights of the ingoing edges form a convolution operation on the basis of a particular number of kernels. In particular, the structure and the weights of the ingoing edges may be selected such that the values

$${x}_{k}^{( n )}$$

of the nodes 29 of the succeeding node layer 32 may be defined as a convolution x⁽ⁿ⁾=K*x⁽ⁿ⁻¹⁾ on the basis of the values x⁽ⁿ⁻¹⁾ of the nodes 28 of the preceding node layer 23, wherein the convolution * in the two-dimensional case may be defined as

$${x}^{( n )} [ i , j ] = ( K * {x}^{( n - 1 )} ) [ i , j ] = {\sum }_{{i}^{′}} {\sum }_{{j}^{′}} K [ {i}^{′} , {j}^{′} ] \cdot {x}^{( n - 1 )} [ i - {i}^{′} , j - {j}^{′} ]$$

Therein, the kernel Kₖ is a d-dimensional matrix, in this example embodiment, a two-dimensional matrix, which is typically small in comparison with the number of the nodes 28, 29, for example, a 3×3 matrix or a 5×5 matrix. In particular, this implies that the weights of the edges are not independent, but rather are selected such that they generate the above convolution equation.

In general, convolutional neural networks 22 use node layers 23, 32, 33 with a plurality of channels, in particular, due to the use of a plurality of kernels in convolutional layers 24. In such cases, the node layers may be understood as a (d+1)-dimensional matrix wherein the first dimension indexes the channels. The effect of a convolutional layer 24 is then defined in a two-dimensional example as

$${x}^{{( n )}_{b}} [ i , j ] = {\sum }_{a} ( {K}_{a , b} * {x}^{{( n - 1 )}_{a}} ) [ i , j ] = {\sum }_{a} {\sum }_{{i}^{′}} {\sum }_{{j}^{′}} {K}_{a , b} [ {i}^{′} , {j}^{′} ] \cdot {x}^{{( n - 1 )}_{a}} [ i - {i}^{′} , j - {j}^{′} ] ,$$

wherein x⁽ⁿ⁻¹⁾ₐ corresponds to the a-th channel of the preceding node layer 23, x⁽ⁿ⁻¹⁾_{b} corresponds to the b-th channel of the succeeding node layer 21 and K_{a,b} corresponds to one of the kernels. If a convolutional layer 24 acts upon a preceding node layer 23 with A channels and outputs a node layer 32 with B channels, then A·B independent d-dimensional kernels K_{a,b} exist.

In general, activation functions are used in convolutional neural networks 22. In this example embodiment, ReLUs (acronym for Rectified Linear Units) are used, wherein R(z)=max(0, z), so that, in the two-dimensional example, the effect of the convolutional layer 24 may be written as

$${x}^{{( n )}_{b}} [ i , j ] = R ( {\sum }_{a} ( {K}_{a , b} * {x}^{{( n - 1 )}_{a}} ) [ i , j ] ) = R ( {\sum }_{a} {\sum }_{{i}^{′}} {\sum }_{{j}^{′}} {K}_{a , b} [ {i}^{′} , {j}^{′} ] \cdot {x}^{{( n - 1 )}_{a}} [ i - {i}^{′} , j - {j}^{′} ] )$$

It is also possible to use other activation functions, for example, ELU (Exponential Linear Unit), LeakyReLU, sigmoid functions, tanh or softmax.

In the example embodiment shown, the input layer 23 comprises thirty six nodes 28 which are arranged in a two-dimensional 6×6 matrix. The first hidden node layer 32 comprises seventy two nodes 29 which are arranged as two two-dimensional 6×6 matrices, wherein each of the two matrices is the result of a convolution of the values of the input layer 23 with a 3×3 kernel in the convolution kernel 24. In the same way, the nodes 29 of the first hidden node layer 32 may be understood as being arranged in a three-dimensional 2×6×6 matrix, wherein the first dimension corresponds to the channel dimension.

The advantage of the use of convolutional layers 24 is that the spatially local correlation of the input data may be utilized in that a local connecting pattern is created between nodes of adjacent layers, in particular in that each node has connections only to a small region of the nodes of the preceding layer.

A pooling layer 25 is a connecting layer between a preceding node layer 32 with node values x⁽ⁿ⁻¹⁾ and a succeeding node layer 33 with node values x⁽ⁿ⁾. A pooling layer 25 may be characterized by the structure and the weights of the edges and the activation function, wherein the activation function carries out a pooling operation on the basis of a non-linear pooling function f. For example, in the two-dimensional case, the values x⁽ⁿ⁾ of the nodes 30 of the succeeding node layer 33 may be calculated on the basis of the values x⁽ⁿ⁺¹⁾ of the nodes 29 of the preceding layer 32 with

$${x}^{{( n )}_{b}} [ i , j ] = f ( {x}^{{( n - 1 )}_{b}} [ {id}_{1} , {jd}_{2} ] , … , {x}^{{( n - 1 )}_{b}} [ ( i + 1 ) {d}_{1} - 1 , ( j + 1 ) {d}_{2} - 1 ] )$$

In other words, by way of the use of a pooling layer 25, the number of nodes 29, 30 may be reduced in that a number d₁·d₂ of adjacent nodes 29 in the preceding layer 32 are replaced by a single node 30 in the succeeding node layer 33 which is calculated as a function of the values of said number of adjacent nodes 29. In particular, the pooling function f may be a maximum function, an averaging function or the L2 norm. In particular, for a pooling layer 25, the weights of the ingoing edges may be specified and not modified by training.

The advantage of the use of a pooling layer 25 is that the number of nodes 29, 30 and the number of parameters is reduced. This leads to a reduction of the required calculation quantity within the convolutional neural network 22 and thus to a control of overfitting.

In the example embodiment shown, the pooling layer 25 is a max pooling layer in which four adjacent nodes are replaced with just one single node, the value of which is formed by the maximum of the values of the four adjacent nodes. The max pooling is applied to each d-dimensional matrix of the preceding layer; in this example embodiment, the max pooling is applied to each of the two two-dimensional matrices so that the number of nodes is reduced from seventy two to eighteen.

In general, the last layers of a convolutional neural network 22 are fully connected layers 26. A fully connected layer 26 is a connecting layer between a preceding node layer 33 and a succeeding node layer 27. A fully connected layer 26 is distinguished in that a plurality, in particular all, of the edges between the nodes 30 of the preceding node layer 33 and the nodes 31 of the succeeding node layer 27 are present, wherein the weight of each of the edges may be individually adapted.

In this example embodiment, the nodes 30 of the preceding node layer 33 and of the succeeding node layer 27 are shown both as two-dimensional matrices and also as non-coherent nodes (shown as one row of nodes, wherein the number of the nodes has been reduced for better clarity). This operation is referred to as “flattening”. In this example embodiment, the number of the nodes 31 in the succeeding node layer 27 of the fully connected layer 26 is smaller than the number of nodes 30 in the preceding node layer 33 of the fully connected layer 26. In alternative embodiments, the number of the nodes 31 may be the same or greater.

Furthermore, in this example embodiment, the softmax function is applied to the fully connected layer 26. By applying the softmax function, the sum of the values of all the nodes 31 of the output layer 27 is one and all the values of all the nodes 31 of the output layer are real numbers between 0 and 1. If the convolutional neural network 22 is used for classifying input data, in particular, the values of the output layer 27 may be interpreted as a probability that the input data falls into one of the different classes.

Convolutional neural networks 22 may be trained, in particular, on the basis of the back-propagation algorithm. In order to prevent an overfitting, regularization methods may be used, for example, dropout of individual nodes 28 to 31, stochastic pooling, the use of synthetic data, weight decay on the basis of the L1 or L2 norm or max-norm constraints.

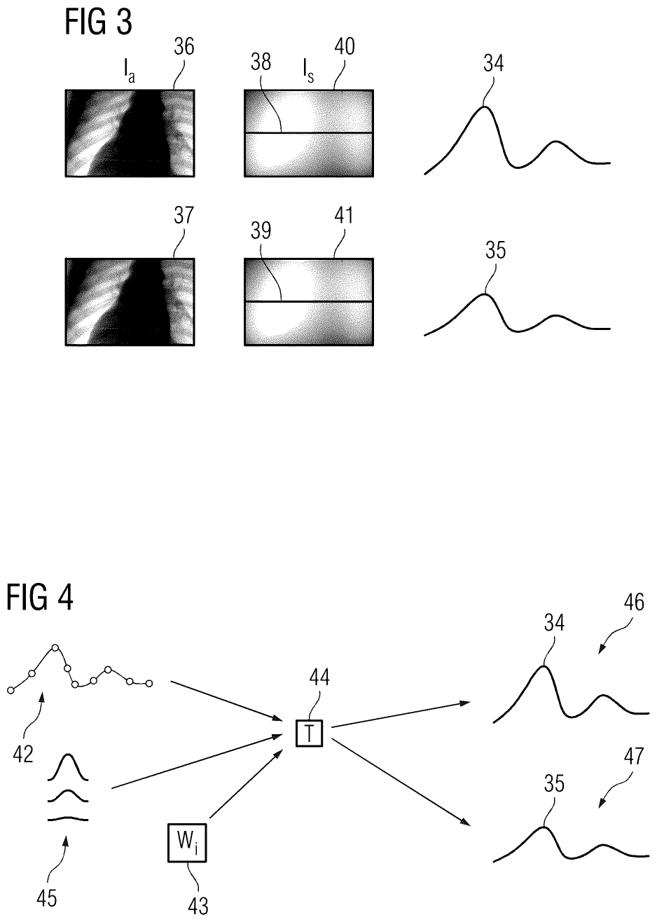

FIG. 3 shows schematically progressions 34, 35 of scattered radiation contributions in two X-ray images 36, 37 which have been recorded with different image recording parameters but the same specified imaging object and the same specified recording geometry. This involves the progression along a line 38, 39 in a scattered radiation image 40, 41, defining the scattered radiation distribution. By way of example, in the present case, the X-ray image 36 has been recorded with a high energy spectrum and the X-ray image 37 with a low energy spectrum. Evidently, there result similar, in the present case substantially scaled, progressions 34, 35. The scattered radiation images 40, 41 also have a similarity, although they are not shown in detail.

On the basis of this finding, it is now possible, as shown schematically in FIG. 2, to describe the scattered radiation distribution for X-ray images 36, 37, each with associated value sets of the image recording parameters by way of an average distribution 42 dependent upon the imaging object and the recording geometry, symbolized herein again, by way of example, as a progression along a line that is part of a vertex-based mesh surface or a surface defined by B-splines, and transformation parameters 43 of a transformation 44 dependent upon the image recording parameters, which in the present case, is described by basic variation components 45 (variation basis components), wherein, in the present case, the transformation parameters 43 are weights for the variation basis components 45. For example, the basic variation components 45 may be displacement vectors that form a basis for the transformation 44. In other representations of the average distribution 42, other forms of basic variation components 45 or of the transformation 44 may be used, as previously described.

If transformation parameters 43 that define the high energy spectrum are used, the result of the transformation 44 is the scattered radiation distribution 46 defined by the scattered radiation image 40 symbolized by the progression 34, whereas if transformation parameters 43 that define the low energy spectrum are used, the result of the transformation 44 is the scattered radiation distribution 47 defined by the scattered radiation image 41, symbolized by the progression 35.

FIG. 5 illustrates the application of this breakdown in an example embodiment of the determining method according to the invention. Therein, as the function set, a first trained evaluating function 48 and a second trained evaluating function 49 are provided which, in the present case, each comprise a CNN 22. The first evaluating function is trained to determine, from an X-ray image 36, 37 as input data, an average distribution dataset 52 defining the associated average distribution 42 of the scattered radiation in the scenario, that is therefore to be used for this scenario of a particular imaging object in a particular recording geometry. The second evaluating function determines, from the values of the image recording parameters 51 of the X-ray image 50 that have been provided with it, the associated transformation parameters 43 of the transformation 44. Therein, apart from the evaluating functions 48, 49, in the present case, the basic variation components 45 of the transformation 44 are also specified, and thus fixed for all scenarios. In principle, it is however also conceivable to include their selection at least partially into the transformation parameters 44 and to determine them by way of the second trained evaluating function 49.

From the determined average distribution dataset 52 and the determined transformation parameters 43, by means of the transformation 44, the corresponding scattered radiation information 53, referred to here as the scattered radiation image 54 may then be determined. This may then be used, for example, for correcting the X-ray image 50 for the corresponding scattered radiation contributions, for example, by subtraction of the scattered radiation image 54.

With the trained evaluating functions 48, 49 and the transformation 44, safety conditions may possibly also be provided, each of which evaluates at least one of the transformation parameters 43 that the second trained evaluating function 49 determines. If a safety condition has been met, this indicates that the corresponding transformation parameter 43 appears to be implausible in respect of the transformation parameter 43 occurring during training. A user may then be notified of this plausibility level by way of a message output. It is additionally possible, for example, after corresponding selection by the user or otherwise, then to use a possibly less accurate but more robust fallback method for determining the scattered radiation information 53.

In this example, the image recording parameters 51 are provided as a spatial distribution of the focal spot and an X-ray spectrum, at least in respect of the X-ray radiator, in this case, an X-ray tube. Thereby, the most extensive physical information possible is provided.

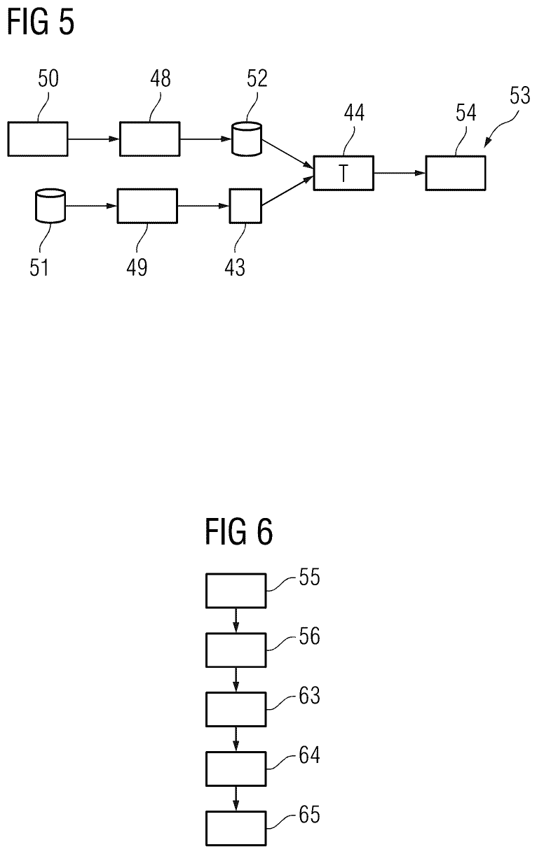

FIG. 6 illustrates an example embodiment of a providing method in order to train the first and the second evaluating function 48, 49 and to provide them together with the transformation 44 and possibly the safety conditions. Therein, in a step 55, the first and second evaluating functions 48, 49 that are to be trained, and training datasets are provided. Each training dataset relates to one of a first number of basic scenarios that are distinguished by a specified imaging object in a specified recording geometry. In each training dataset, that is, for each basic scenario, there exist subdatasets, each comprising a value set of image recording parameters, a training X-ray image recorded in the basic scenario with the value set and an associated training scattered radiation image which defines the scattered radiation distribution in the basic scenario with the value set. The value sets of the operating parameters therein ideally cover a settable range of each of the operating parameters as well as possible and also contain, in particular, extreme values of the operating parameters that are settable. The second number of subdatasets may be, for example, fifty.

The training data, that is, in particular, the training X-ray images and the training scattered radiation images have been determined, in this case, by simulation; in principle, however, an at least partial measurement, for example, using a phantom, is conceivable.

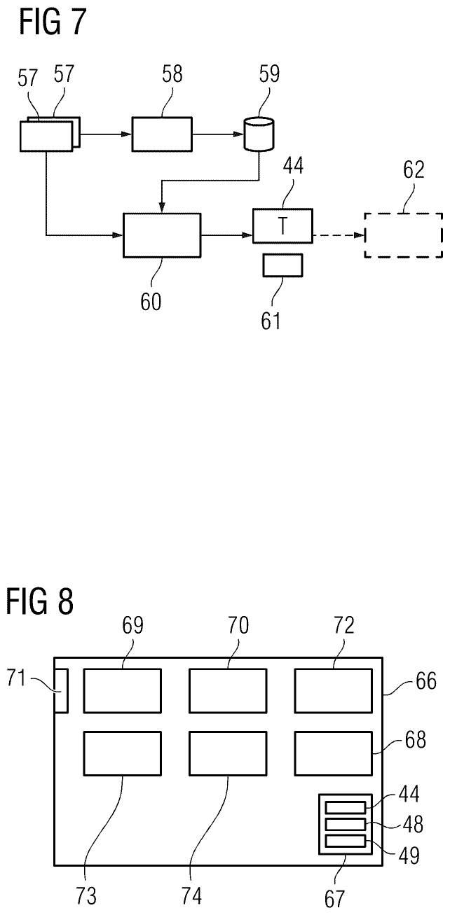

In one step 56, the training datasets are evaluated in order to determine training average distribution datasets as ground truths for the first evaluating function 48, the transformation 44 and training transformation parameters as ground truths for the second evaluating function 49. This procedure is described in more detail making reference to FIG. 7.

The training scattered radiation images 57 of a basic scenario are initially used in a substep 58 in order to determine a training average distribution dataset 59 for this basic scenario by average formation, generalized Procrustes analysis or suchlike. This takes place for each basic scenario. Thereafter, the training scattered radiation images 57 are used for all the basic scenarios and the training average distribution datasets 59 are used for all the basic scenarios in order to determine the transformation 44 and the training transformation parameters 61 in a subset 60. An optional harmonization step 62 may provide that the transformation 44, in particular therefore the basic variation components 45 are the same for all the basic scenarios. This is suitable, in particular, if the substep 60 operates in the manner of a basic scenario so that for different basic scenarios, different bases may come about for the transformation 44. For example, the basic variation components 45 may then be averaged for the basic scenarios and the transformation parameters, which form image-specific weights, may be adapted accordingly.

In the subset 60, for example, principal component analysis (PCA), independent component analysis (ICA), a variational autoencoder (VAE) or related techniques may be utilized.

In a step 63, the first and the second evaluating functions 48, 49 are then separately trained under supervision with the respective training input data (training X-ray images, training value sets of the image recording parameters 51) and the respective training output data (training average distribution dataset 59, training transformation parameters 61).

It should be noted at this point that, in principle, other possibilities for using the training datasets also exist in order to train the first and the second evaluating functions 48, 49, possibly semi-supervised. For example, it is conceivable also to determine the average distribution of the scattered radiation for the basic scenarios implicitly in the context of the training of the first evaluating function 48 in that all the training X-ray images are combined with all the training scattered radiation images 57 (as the training average distribution datasets) and thereby the averaging is carried out implicitly. It is also conceivable, after the training of one of the evaluating functions 48, 49 to keep it constant and to train the other evaluating function 49, 48 in the overall process. Furthermore, the transformation 44, in particular, the basic variation components 45 may be made part of the training process of the second evaluating function 49. Reference is made to the description of these variants already set out above.

In an optional step 64, the at least one safety condition may be determined. For this purpose, a statistical value, in particular, an extreme value and/or another statistically relevant value (for example, mean value, standard deviation, median) is determined for at least one of the transformation parameters 43, 61 as they occur in the training. They may be used, for example, by way of comparisons in the safety conditions during the inference in order to identify implausible or improbable weights, that is, transformation parameters 43, as already described above.

In a step 65, the trained first evaluating function 48, the trained second evaluating function 49, the transformation 44 and possibly the safety conditions are provided, for example, in order to be applied as described in relation to FIG. 5.

In all the example embodiments described, the scattered radiation image 54 belonging to the X-ray image 50 may be used for correcting the X-ray image 50 for scattered radiation contributions.

FIG. 8 shows a sketch of the principle of an evaluating facility 66 according to the present embodiments. The evaluating facility 66 comprises a storage means 67 in which the transformation 44, in particular, the basic variation components 45, the first trained evaluating function 48 and the second trained evaluating function 49 are provided. Optionally, the evaluating facility 66 may have a training unit 68 for carrying out the training process described in relation to FIG. 6, so that then a providing facility is integrated into the evaluating facility 66.

The evaluating facility 66 further has a first applying unit 69 for applying the first trained evaluating function 48 to the X-ray image 50. In a second applying unit 70, the second trained evaluating function 49 is applied to the image recording parameters 51 of the X-ray image 50. Both the X-ray image 50 and also the values of the image recording parameter 51 that are associated with it may have been provided via an interface 71. In a determining unit 72, by means of the average distribution dataset 52 determined by the first applying unit 69, the transformation parameters 43 determined by the second applying unit 70 and the transformation 44, the scattered radiation image 54 associated with the X-ray image 50 is determined.

Optionally, a correcting unit 73 is also provided in order to carry out the described correction of the X-ray image 50 with the scattered radiation image 54. Furthermore, a monitoring unit 74 may be provided to check the fulfilment of the at least one safety condition.

The evaluating facility 66 is therefore configured to carry out a determining method according to the present embodiments.

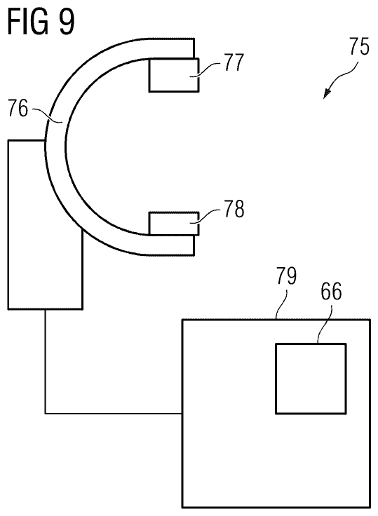

Finally, FIG. 9 shows a sketch of the principle of an imaging X-ray facility 75, here comprising a C-arm 76 on which an X-ray radiator 77 (here an X-ray tube) and an X-ray detector 78 are arranged opposite one another. For example, the X-ray facility 75 may be an angiography facility, in particular, for neurovascular imaging. Using the X-ray facility 75, both two-dimensional X-ray images for diagnostics and/or intervention monitoring, in particular, therefore radiographs and/or fluoroscopic images may be recorded, as well as cone beam computed tomography in that projection images are recorded from different projection directions, from which a three-dimensional image dataset is reconstructed. The X-ray facility 75 has no anti-scatter grid on the X-ray detector 78.

In order to be able to correct at least the projection images of the CBCT, and possibly also other two-dimensional X-ray images, for scattered radiation contributions, an evaluating facility 66 according to the present embodiments is integrated into a control facility 79 controlling the operation of the X-ray facility 75.

Independent of the grammatical term usage, individuals with male, female or other gender identities are included within the term.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for providing a trained function set for determining a scattered radiation information item defining a scattered radiation contribution in an X-ray image that is two-dimensional and is recorded with an X-ray facility, the method being computer-implemented and comprising:
providing a first evaluating function that is to be trained, that determines, from input data comprising the X-ray image, an average distribution dataset that defines an average scattered radiation distribution over a variation of at least one image recording parameter, as output data;
providing a second evaluating function that is to be trained that determines, from input data comprising the at least one image recording parameter during recording of the X-ray image, transformation parameters of a transformation as output data so that the scattered radiation information item is determinable by applying the transformation to the average distribution dataset;
providing a training dataset for each of a first number of basic scenarios for which only the at least one image recording parameter is changeable, wherein each training dataset for different values of the at least one image recording parameter comprises a second number of subdatasets, each comprising a training X-ray image as input data for the first evaluating function, a training value set of the at least one image recording parameter as input data for the second evaluating function and a training scattered radiation image comprising the scattered radiation distribution as a respective ground truth;
separate training of the first evaluating function and the second evaluating function, making use of the training datasets; and
providing the trained first evaluating function and the trained second evaluating function.

2. The method of claim 1, wherein the basic scenarios relate to the recording of a specified imaging object in a specified recording geometry, the at least one image recording parameter is selected from the group comprising a tube current of an X-ray radiator of the X-ray facility, a tube voltage of the X-ray radiator, a focal spot size and a type of focus of the focal spot of the X-ray radiator, at least one filter parameter defining a filter measure for the X-ray radiation, and at least one modulation parameter relating to a modulation of the X-ray radiation.

3. The method of claim 1, wherein the image recording parameters are provided as an X-ray spectrum of the X-ray radiation, a spatial distribution for modeling the focal spot, or the X-ray spectrum of the X-ray radiation and the spatial distribution for modeling the focal spot.

4. The method of claim 1, wherein training output datasets are determined as a ground truth for training at least one of the evaluating functions from the training datasets that are used for supervised learning of the respective evaluating function.

5. The method of claim 4, wherein for each basic scenario, a training average distribution dataset is determined from the scattered radiation distributions that are defined by the training scattered radiation images of the subdatasets, via average formation, generalized Procrustes analysis, or average formation and generalized Procrustes analysis, and
wherein the first evaluating function is trained with corresponding training X-ray images of all the subdatasets of all the training datasets.

6. The method of claim 4, wherein when the output average distribution datasets are determined for each basic scenario, the transformation and the training transformation parameters to be used as a ground truth are determined from the output average distribution datasets and the training scattered radiation images.

7. The method of claim 1, wherein during the training, at least one statistical value defining the distribution of the transformation parameters is determined per transformation parameter, from which statistical value at least one safety condition for plausibility checking of the transformation parameters that are output by the second evaluating function is determined and is provided therewith.

8. The method of claim 1, wherein the first evaluating function is trained in terms of basic scenarios, with all the conceivable pairings of training X-ray images and training scattered radiation images as a ground truth.

9. The method of claim 1, wherein in a transformation comprising the weighted application of basic variation components in the training process for the second evaluating function, the basic variation components are also used as trainable parameters.

10. The method of claim 9, wherein the transformation is modeled as a plurality of trained transformation functions, and
wherein each basic variation component is associated with exactly one trained transformation function.

11. The method of claim 10, wherein the plurality of trained transformation function are neural networks.

12. A method for determining a scattered radiation information item defining a scattered radiation contribution in a two-dimensional X-ray image recorded with an X-ray facility, the method being computer-implemented and comprising:
providing a trained function set using a method for providing a trained function set for determining a scattered radiation information item defining a scattered radiation contribution in an X-ray image that is two-dimensional and is recorded with an X-ray facility, the method for providing the trained function set being computer-implemented and comprising:
providing a first evaluating function that is to be trained, that determines, from input data comprising the X-ray image, an average distribution dataset that defines an average scattered radiation distribution over a variation of at least one image recording parameter, as output data;
providing a second evaluating function that is to be trained that determines, from input data comprising the at least one image recording parameter during recording of the X-ray image, transformation parameters of a transformation as output data so that the scattered radiation information item is determinable by applying the transformation to the average distribution dataset;
providing a training dataset for each of a first number of basic scenarios for which only the at least one image recording parameter is changeable, wherein each training dataset for different values of the at least one image recording parameter comprises a second number of subdatasets, each comprising a training X-ray image as input data for the first evaluating function, a training value set of the at least one image recording parameter as input data for the second evaluating function and a training scattered radiation image comprising the scattered radiation distribution as a respective ground truth;
separate training of the first evaluating function and the second evaluating function, making use of the training datasets; and
providing the trained first evaluating function and the trained second evaluating function, wherein the function set comprises:
a first trained evaluating function that determines, from input data comprising the X-ray image, an average distribution dataset that defines an average scattered radiation distribution over a variation of at least one image recording parameter, as output data; and
a second trained evaluating function that determines, from input data comprising the at least one image recording parameter during recording of the X-ray image, transformation parameters of a transformation as output data so that the scattered radiation information item is determinable by applying the transformation to the average distribution dataset;
providing the X-ray image to be evaluated and the at least one image recording parameter associated with the X-ray image;
applying the first trained evaluating function to the X-ray image to be evaluated as input data for determining an average distribution dataset associated with the X-ray image to be evaluated, as output data;
applying the second trained evaluating function to the at least one image recording parameter associated with the X-ray image to be evaluated, as input data for determining transformation parameters associated with the X-ray image to be evaluated, as output data; and
determining the scattered radiation information item, the determining of the scattered radiation information item comprising applying the transformation according to the transformation parameters associated with the X-ray image to be evaluated, to the average distribution dataset associated with the X-ray image to be evaluated.

13. The method of claim 12, wherein determining the scattered radiation information item comprises determining the scattered radiation information item in the form of a scattered radiation image.

14. The method of claim 12, wherein for a subsequent recording process with the same imaging object and the same recording geometry, in an optimizing process making use of the second evaluating function with variation of the at least one image recording parameter, an optimum set of the at least one image recording parameter is determined for a predetermined optimization goal.

15. An evaluating facility for determining a scattered radiation information item defining a scattered radiation contribution in a two-dimensional X-ray image recorded with an X-ray facility, the evaluating facility comprising:
a storage device in which a trained function set is stored, wherein the function set comprises:
a first trained evaluating function that determines, from input data comprising the X-ray image, an average distribution dataset that defines an average scattered radiation distribution over a variation of at least one image recording parameter, as output data;
a second trained evaluating function that determines, from input data comprising the at least one image recording parameter during recording of the X-ray image, transformation parameters of a transformation as output data so that the scattered radiation information item is determinable by applying the transformation to the average distribution dataset;
an interface for receiving the X-ray image to be evaluated and the at least one image recording parameter associated with the X-ray image;
a first applying unit for applying the first trained evaluating function to the X-ray image to be evaluated, as input data for determining an average distribution dataset associated with the X-ray image to be evaluated, as output data;
a second applying unit for applying the second trained evaluating function to the at least one image recording parameter associated with the X-ray image to be evaluated, as input data for determining transformation parameters associated with the X-ray image to be evaluated, as output data; and
a determining unit configured to determine the scattered radiation information item in the form of a scattered radiation image by application of the transformation according to the transformation parameters associated with the X-ray image to be evaluated, to the average distribution dataset associated with the X-ray image to be evaluated.

16. In a non-transitory computer-readable storage medium that stores instructions executable by one or more processors to provide a trained function set for determining a scattered radiation information item defining a scattered radiation contribution in an X-ray image that is two-dimensional and is recorded with an X-ray facility, the instructions comprising:
providing a first evaluating function that is to be trained, that determines, from input data comprising the X-ray image, an average distribution dataset that defines an average scattered radiation distribution over a variation of at least one image recording parameter, as output data;
providing a second evaluating function that is to be trained that determines, from input data comprising the at least one image recording parameter during recording of the X-ray image, transformation parameters of a transformation as output data so that the scattered radiation information item is determinable by applying the transformation to the average distribution dataset;
providing a training dataset for each of a first number of basic scenarios for which only the at least one image recording parameter is changeable, wherein each training dataset for different values of the at least one image recording parameter comprises a second number of subdatasets, each comprising a training X-ray image as input data for the first evaluating function, a training value set of the at least one image recording parameter as input data for the second evaluating function and a training scattered radiation image comprising the scattered radiation distribution as a respective ground truth;
separate training of the first evaluating function and the second evaluating function, making use of the training datasets; and
providing the trained first evaluating function and the trained second evaluating function.
